(19) United States
(12) Patent Application Publication
(10) Pub. No.: US 2004/0102469 A1
(43) Pub. Date: May 27, 2004

(54) Method for reducing the mortality rate

(75) Inventors: Bernd Disse, Mainz am Rhein (DE); Steven Kesten, Southbury, CT (US); Theodore Witek, Oberursel (DE)

Correspondence Address: BOEHRINGER INGELHEIM CORPORATION, 900 RIDGEBURY ROAD, P. O. BOX 368, RIDGEFIELD, CT, 06877, US

(73) Assignees: Boehringer Ingelheim Pharma GmbH & Co. KG, Ingelheim (DE); Boehringer Ingeilheim Pharmaceuticals, Inc., Ridgefield, CT

(21) Appl. No.: 10/640,887

(22) Filed: Aug. 14, 2003

Related U.S. Application Data
(60) Provisional application No. 60/410,623, filed on Sep. 13, 2002.

(51) Int. Cl. A61K 31/4745

(52) U.S. Cl. 514/291

(57) ABSTRACT

A method for reducing the mortality rate in a patient comprising administering to the patient a therapeutically effective amount of a tiotropium salt.

### RELATED APPLICATIONS

This application claims priority to U.S. Serial No. 60/410,623, filed Sep. 13, 2002, which is hereby incorporated by reference in its entirety.

### BACKGROUND OF THE INVENTION

The invention relates to a method for the reduction of the mortality rate in patients with lung diseases, the method comprising the administration of a therapeutically effective amount of (1α,2β,4β,5α,7β)-7-[(hydroxydi-2-thienylacetyl)oxy]-9,9-dimethyl-3-oxa-9-azoniatricyclo[3.3.1.0^{2,4}]nonane salts.

Antimuscarinics which are also often referred to in clinical practice as anticholinergics are firmly established in the treatment of diseases of the respiratory tract. For example, the administration of ipratropium bromide by inhalation (e.g., sold under the trademark ATROVENT®) as a bronchodilator is an established standard for the treatment of obstructive lung diseases, especially COPD, a term used hereinafter to refer to the related syndromes of chronic bronchitis, chronic obstructive bronchitis, and pulmonary emphysema. Anticholinergics are also used to treat asthma on account of their bronchodilatory effect.

The compound (1α,2β,4β,5α,7β)-7-[(hydroxydi-2-thienylacetyl)oxy]-9,9-dimethyl-3-oxa-9-azoniatricyclo[3.3.1.0^{2,4}]nonane bromide is known from European Patent Application EP 418 716 A1 (corresponding to U.S. Pat. No. 5,610,165, hereby incorporated by reference) and has the following chemical structure:

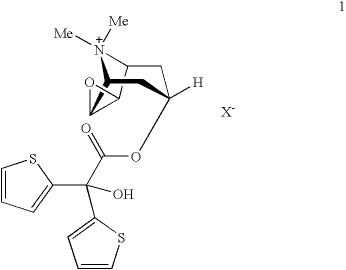

where X denotes bromide. The term tiotropium should be taken as being a reference to the free cation (1′) within the scope of the present invention.

Tiotropium bromide, as well as other salts of tiotropium, are known as highly effective anticholinergic bronchodilators.

Tiotropium salts 1 are preferably administered by inhalation. Suitable inhalable powders packed into appropriate capsules (inhalettes) and administered using suitable powder inhalers may be used. Alternatively, they may be administered by the use of suitable inhalable aerosols. These also include powdered inhalable aerosols which contain, for example, HFA134a, HFA227, or mixtures thereof as propellant gas. The preparations may also be inhaled in the form of suitable solutions of the tiotropium salt 1.

### DESCRIPTION OF THE INVENTION

Surprisingly, the antimuscarinically active tiotropium salts 1 can not only be used effectively as bronchodilators, but are also suitable in the reduction of the mortality rate. This is evidence by the number of people with lung diseases, especially COPD, who die from respiratory disease in a given prolonged period of time when regularly self-administering tiotropium through the HANDIHALER® inhalation device; the comparison being a population with similar characteristics not treated with tiotropium.

The finding that salts of formula 1 are useful in the reduction of the mortality rate is surprising, because the predecessor molecule ipratropium bromide in a five-year study did not show any influence on mortality. The obvious difference between ipratropium and tiotropium is the much longer duration of action of the latter molecule, however, this difference is not an explanation for an influence on mortality according to present state of the art.

The invention therefore relates to a method for reducing the mortality rate in patients comprising the administration of a therapeutically effective amount of tiotropium salts 1.

Preferably, the invention relates to a method for reducing the respiratory mortality rate in patients suffering from lung diseases comprising the administration of a therapeutically effective amount of tiotropium salts 1.

More preferably, the invention relates to a method for reducing the respiratory mortality rate in patients suffering from obstructive and restrictive lung disease, for example, bronchiectasis, cystic fibrosis, chronic bronchitis, chronic obstructive bronchitis, asthma, chronic obstructive pulmonary disease (COPD), emphysema and forms of fibrosing lung diseases comprising the administration of a therapeutically effective amount of tiotropium salts 1.

In particular, the invention relates to a method for reducing respiratory mortality in patients suffering from chronic (obstructive) bronchitis and COPD with and without emphysema.

By the tiotropium salts 1 which may be used within the scope of the present invention are meant the compounds which contain, in addition to tiotropium as counter-ion (anion), chloride, bromide, iodide, methanesulfonate, p-toluenesulfonate, or methylsulfate. Within the scope of the present invention, the methanesulfonate, chloride, bromide, and iodide are preferred of all the tiotropium salts, the methanesulfonate and bromide being of particular importance. Tiotropium bromide is of outstanding importance according to the invention.

In another aspect, the present invention relates to pharmaceutical preparations for treating the abovementioned diseases. Without restricting the scope of the invention thereto, these may contain tiotropium 1′ in amounts such that each individual dose contains 0.1 μg to 80 μg, preferably 0.5 μg to 60 μg, most preferably about 1 μg to 50 μg. For example, and without restricting the scope of the invention thereto, 2.5 μg, 5 μg, 10 μg, 18 μg, 20 μg, 36 μg, or 40 μg of 1′ may be administered per single dose.

If tiotropium bromide is used as the preferred tiotropium salt 1 according to the invention, the amounts of active substance 1′ administered per single dose as specified hereinbefore by way of example correspond to the following amounts of 1 administered per single dose: 3 μg, 6 μg, 12 μg, 21.7 μg, 24.1 μg, 43.3 μg, and 48.1 μg of 1.

Use of tiotropium salts 1 according to the invention includes the use of the solvates and hydrates thus formed, preferably the hydrates, most preferably the monohydrates. Of particular interest within the scope of the invention is the aforementioned method comprising the administration of the crystalline tiotropium bromide monohydrate as disclosed in WO 02/30928 (corresponding to U.S. pat. app. Pub. No. 2002/0169321, which is hereby incorporated by reference).

If, for example, tiotropium bromide monohydrate is used as the preferred tiotropium salt 1 according to the invention, the amounts of active substance 1′ administered per single dose as specified hereinbefore by way of example correspond to the following amounts of 1 (monohydrate) administered per single dose: 3.1 μg, 6.2 μg, 12.5 μg, 22.5 μg, 25 μg, 45 μg, and 50 μg.

The tiotropium salts 1 are preferably administered according to the invention by inhalation. For this purpose, the tiotropium salts 1 have to be prepared in inhalable forms. Inhalable preparations include inhalable powders, propellant-containing metering aerosols or propellant-free inhalable solutions. Inhalable powders according to the invention containing the tiotropium salts 1 are optionally mixed with physiologically acceptable excipients. Within the scope of the present invention, the term propellant-free inhalable solutions also includes concentrates or sterile inhalable solutions ready for use. The formulations which may be used within the scope of the present invention are described in more detail in the next part of the specification.

### A) Inhalable Powder

The inhalable powders which may be used according to the invention may contain 1 either on its own or in admixture with suitable physiologically acceptable excipients.

If the tiotropium salts 1 are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, or maltose), oligo- and polysaccharides (e.g., dextrane), polyalcohols (e.g., sorbitol, mannitol, or xylitol), salts (e.g., sodium chloride or calcium carbonate), or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 μm and 150 μm, most preferably between 15 μm and 80 μm. It may sometimes seem appropriate to add finer excipient fractions with an average particle size of 1 μm to 9 μm to the excipients mentioned above. These finer excipients are also selected from the group of possible excipients listed hereinbefore. Finally, in order to prepare the inhalable powders according to the invention, micronized active substance 1, preferably with an average particle size of 0.5 μm to 10 μm, more preferably from 1 μm to 6 μm, is added to the excipient mixture. Processes for producing the inhalable powders according to the invention by grinding and micronizing and by finally mixing the ingredients together are known from the prior art.

Particular preferred inhalable powders applicable within the scope of the instant invention are those disclosed in WO 02/30389.

Inhalable powders according to the invention which contain a physiologically acceptable excipient in addition to 1 may be administered, for example, by means of inhalers which deliver a single dose from a supply using a measuring chamber as described in U.S. Pat. No. 4,570,630, which is hereby incorporated by reference, or by other means as described in DE 36 25 685 A. The inhalable powders according to the invention which contain 1 optionally in conjunction with a physiologically acceptable excipient may be administered for example using an inhaler known by the name TURBUHALER® or using inhalers as disclosed, for example, in EP 237507 A. Preferably, the inhalable powders according to the invention which contain physiologically acceptable excipient in addition to 1 are packed into capsules (to produce so-called inhalettes) which are used in inhalers as described, for example, in WO 94/28958 (corresponding to U.S. Pat. No. 5,947,118, which is hereby incorporated by reference).

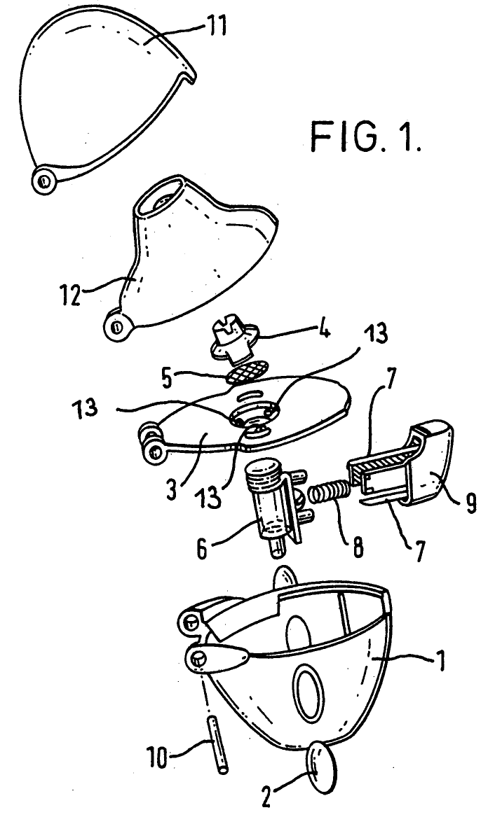

A particularly preferred inhaler for administering the pharmaceutical combination according to the invention in inhalettes is shown in FIG. 1.

The inhaler according to FIG. 1 is characterized by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, a mouthpiece 12 which is connected to the housing 1, the deck 3 and a cover 11 via a spindle 10 to enable it to be flipped open or shut and three holes 13 with diameters below 1 mm in the central region around the capsule chamber 6 and underneath the screen housing 4 and screen 5.

The main air flow enters the inhaler between deck 3 and base 1 near to the hinge. The deck has in this range a reduced width, which forms the entrance slit for the air. Then the flow reverses and enters the capsule chamber 6 through the inlet tube. The flow is then further conducted through the filter and filter holder to the mouthpiece. A small portion of the flow enters the device between mouthpiece and deck and flows then between filter holder and deck into the main stream. Due to production tolerances, there is some uncertainty in this flow because of the actual width of the slit between filter holder and deck. In case of new or reworked tools, the flow resistance of the inhaler may therefore be a little off the target value. To correct this deviation, the deck has in the central region around the capsule chamber 6 and underneath the screen housing 4 and screen 5 three holes 13 with diameters below 1 mm. Through these holes 13 flows air from the base into the main air stream and reduces such slightly the flow resistance of the inhaler. The actual diameter of these holes 13 can be chosen by proper inserts in the tools so that the mean flow resistance can be made equal to the target value.

If the inhalable powders according to the invention are packed into capsules (inhalers) for the preferred use described above, the quantities packed into each capsule should be 1 mg to 30 mg, preferably 3 mg to 20 mg, more particularly 5 mg to 10 mg of inhalable powder per capsule. These capsules contain, according to the invention, either together or separately, the doses of 1′ mentioned hereinbefore for each single dose.

### B) Propellant Gas-Driven Inhalation Aerosols

Inhalation aerosols containing propellant gas which may be used according to the invention may contain substances 1 dissolved in the propellant gas or in dispersed form. The propellant gases which may be used to prepare the inhalation aerosols are known from the prior art. Suitable propellant gases are selected from among hydrocarbons such as n-propane, n-butane, or isobutane and halohydrocarbons such as preferably fluorinated derivatives of methane, ethane, propane, butane, cyclopropane, or cyclobutane. The propellant gases mentioned above may be used on their own or in mixtures thereof. Particularly preferred propellant gases are fluorinated alkane derivatives selected from TG134a (1,1,1,2-tetrafluoroethane), TG227 (1,1,1,2,3,3,3-heptafluoropropane), and mixtures thereof.

The propellant-driven inhalation aerosols which may be used according to the invention may also contain other ingredients such as co-solvents, stabilizers, surfactants, antioxidants, lubricants, and pH adjusters. All these ingredients are known in the art.

The propellant-driven inhalation aerosols which may be used according to the invention may contain up to 5 wt. % of active substance 1. The propellant-driven inhalation aerosols which may be used according to the invention contain, for example, 0.002 to 5 wt. %, 0.01 to 3 wt. %, 0.015 to 2 wt. % of active substance 1.

If the active substances 1 are present in dispersed form, the particles of active substance preferably have an average particle size of up to 10 μm, preferably from 0.1 μm to 5 μm, more preferably from 1 μm to 5 μm.

The propellant-driven inhalation aerosols according to the invention which may be used according to the invention may be administered using metered dose inhalers (MDIs) known in the art. Accordingly, in another aspect, the present invention relates to the use of 1 according to the invention to prepare pharmaceutical compositions in the form of propellant-driven aerosols as hereinbefore described combined with one or more inhalers suitable for administering these aerosols.

In addition, the present invention relates to the use of 1 according to the invention to prepare cartridges which when fitted with a suitable valve can be used in a suitable inhaler and which contain one of the abovementioned propellant gas-containing inhalation aerosols according to the invention. Suitable cartridges and methods of filling these cartridges with the inhalable aerosols containing propellant gas according to the invention are known from the prior art.

### C) Propellant-Free Inhalable Solutions

It is particularly preferred to use the tiotropium salts 1 according to the invention to prepare propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulfuric acid, and/or phosphoric acid. Examples of particularly suitable organic acids include ascorbic acid, citric acid, malic acid, tartaric acid, maleic acid, succinic acid, fumaric acid, acetic acid, formic acid, and/or propionic acid etc. Preferred inorganic acids are hydrochloric and sulfuric acids. It is also possible to use the acids which have already formed an acid addition salt with one of the active substances. Of the organic acids, ascorbic acid, fumaric acid, and citric acid are preferred. If desired, mixtures of the above acids may be used, particularly in the case of acids which have other properties in addition to their acidifying qualities, e.g., as flavorings, antioxidants, or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/100 mL, more preferably less than 20 mg/100 mL. Generally, inhalable solutions in which the content of sodium edetate is from 0 to 10 mg/100 mL are preferred.

Co-solvents and/or other excipients may be added to the propellant-free inhalable solutions which may be used according to the invention. Preferred co-solvents are those which contain hydroxyl groups or other polar groups, e.g., alcohols, particularly isopropyl alcohol, glycols, particularly propyleneglycol, polyethyleneglycol, polypropyleneglycol, glycol ether, and glycerol, and polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants, and/or preservatives which guarantee or prolong the shelf life of the finished pharmaceutical formulation, flavorings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols, and similar vitamins and provitamins occurring in the human body.

Preservatives may be used to protect the formulation from contamination with pathogens. Suitable preservatives are those which are known in the art, particularly cetyl pyridinium chloride, benzalkonium chloride, or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the tiotropium salts 1, only benzalkonium chloride and sodium edetate. In another preferred embodiment, no sodium edetate is present.

The propellant-free inhalable solutions which may be used within the scope of the invention are administered in particular using inhalers of the kind which are capable of nebulizing a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 μL and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 (corresponding to U.S. Pat. No. 5,497,944, which is hereby incorporated by reference) and also in WO 97/12687 (corresponding to U.S. Pat. No. 5,964,416, which is hereby incorporated by reference) (cf. in particular FIGS. 6a and 6b). The nebulizers (devices) described therein are also known by the name RESPIMAT®.

This RESPIMAT® nebulizer can advantageously be used to produce the inhalable aerosols according to the invention containing the tiotropium salts 1. Because of its cylindrical shape and handy size of less than 9 cm to 15 cm long and 2 cm to 4 cm wide, this device can be carried at all times by the patient. The nebulizer sprays a defined volume of pharmaceutical formulation using high pressures through small nozzles so as to produce inhalable aerosols.

The preferred atomizer essentially consists of an upper housing part, a pump housing, a nozzle, a locking mechanism, a spring housing, a spring, and a storage container, characterized by:

a pump housing which is secured in the upper housing part and which comprises at one end a nozzle body with the nozzle or nozzle arrangement,

a hollow plunger with valve body,

a power takeoff flange in which the hollow plunger is secured and which is located in the upper housing part,

a locking mechanism situated in the upper housing part,

a spring housing with the spring contained therein, which is rotatably mounted on the upper housing part by means of a rotary bearing, and

a lower housing part which is fitted onto the spring housing in the axial direction.

The hollow plunger with valve body corresponds to a device disclosed in WO 97/12687 (corresponding to U.S. Pat. No. 5,964,416). It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 MPa to 60 MPa (about 50 bar to 600 bar), preferably 10 MPa to 60 MPa (about 100 bar to 600 bar) on the fluid, the measured amount of active substance solution, at its high pressure end at the moment when the spring is actuated. Volumes of 10 to 50 microliters are preferred, while volumes of 10 to 20 microliters are particularly preferred and a volume of 15 microliters per spray is most particularly preferred.

The valve body is preferably mounted at the end of the hollow plunger facing the valve body.

The nozzle in the nozzle body is preferably microstructured, i.e. produced by microtechnology. Microstructured valve bodies are disclosed, for example, in WO 94/07607 (corresponding to U.S. Pat. No. 5,911,851, which is hereby incorporated by reference); reference is hereby made to the contents of this specification, particularly FIG. 1 therein and the associated description.

The valve body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20° to 160° to one another, preferably 60° to 150°, most preferably 80° to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 bar to 300 bar, and is atomized into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annual plane. Details of the construction of the locking mechanism are given in WO 97/20590 (corresponding to U.S. Pat. No. 6,453,795, which is hereby incorporated by reference).

The lower housing part is pushed axially over the spring housing and covers the mounting, the drive of the spindle and the storage container for the fluid.

When the atomizer is actuated, the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360°, e.g., 180°. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention.

The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of construction are disclosed in PCT Applications WO 97/12683 (corresponding to U.S. Pat. No. 6,176,442, which is hereby incorporated by reference) and WO 97/20590 (corresponding to U.S. Pat. No. 6,176,442), to which reference is hereby made.

The components of the atomizer (nebulizer) are made of a material which is suitable for its purpose. The housing of the atomizer and, if its operation permits, other parts as well are preferably made of plastics, e.g., by injection molding. For medicinal purposes, physiologically safe materials are used.

FIGS. 6a/b of WO 97/12687 show the RESPIMAT® nebulizer which can advantageously be used for inhaling the aqueous aerosol preparations according to the invention.

FIG. 6a (WO 97/12687) shows a longitudinal section through the atomizer with the spring biased while FIG. 6b (WO 97/12687) shows a longitudinal section through the atomizer with the spring relaxed.

The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end, the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring, the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution). The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations which may be used according to the invention to produce an aerosol suitable for inhalation.

If the propellant-free inhalable solutions which may be used according to the invention are nebulized using the method described above (RESPIMAT® nebulizer) the quantity delivered should correspond to a defined quantity with a tolerance of not more than 25%, preferably 20% of this amount in at least 97%, preferably at least 98% of all operations of the inhaler (spray actuations). Preferably, between 5 mg and 30 mg of formulation, most preferably between 5 mg and 20 mg of formulation are delivered as a defined mass on each actuation.

However, the propellant-free inhalable solutions which may be used according to the invention may also be nebulized by means of inhalers other than those described above, e.g., jet stream inhalers or other stationary nebulizers.

Accordingly, in a further aspect, the invention relates to the method according to the invention administering tiotropium salts 1 in the form of propellant-free inhalable solutions or suspensions as described above combined with a device suitable for administering these formulations, preferably in conjunction with the RESPIMAT® nebulizer. Preferably, the invention relates to the use according to the invention of compounds 1 for preparing propellant-free inhalable solutions or suspensions characterized in that they contain 1 in conjunction with the RESPIMAT® nebulizer. In addition, the present invention relates to the use according to the invention of the above-mentioned devices for inhalation, preferably the RESPIMAT® nebulizer, characterized in that they contain the propellant-free inhalable solutions or suspensions according to the invention as described hereinbefore.

The propellant-free inhalable solutions or suspensions which may be used within the scope of the invention may take the form of concentrates or sterile inhalable solutions or suspensions ready for use, as well as the abovementioned solutions and suspensions designed for use in a RESPIMAT® nebulizer. Formulations ready for use may be produced from the concentrates, for example, by the addition of isotonic saline solutions. Sterile formulations ready for use may be administered using energy-operated fixed or portable nebulizers which produce inhalable aerosols by means of ultrasound or compressed air by the Venturi principle or other principles.

Accordingly, in another aspect, the present invention relates to the use according to the invention of 1 in the form of propellant-free inhalable solutions or suspensions as described hereinbefore which take the form of concentrates or sterile formulations ready for use, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example.

### EXAMPLES OF FORMULATIONS

### A) Inhalable Powders

1)

| Ingredients | μg per capsule |
|---|---|
| tiotropium bromide | 10.8 |
| lactose | 4989.2 |
| Total | 5000 |

2)

| Ingredients | μg per capsule |
|---|---|
| tiotropium bromide | 21.7 |
| lactose | 4978.3 |
| Total | 5000 |

3)

| Ingredients | μg per capsule |
|---|---|
| tiotropium bromide × H₂O | 22.5 |
| lactose | 4977.5 |
| Total | 5000 |

### B) Inhalable Aerosols Containing Propellant Gas

1) Suspension Aerosol

| Ingredients | wt. % |
|---|---|
| tiotropium bromide | 0.015 |
| soya lecithin | 0.2 |
| TG134a:TG227 (2:3) | to 100 |

2) Suspension Aerosol

| Ingredients | wt. % |
|---|---|
| tiotropium bromide | 0.029 |
| absolute ethanol | 0.5 |
| isopropyl myristate | 0.1 |
| TG 227 | to 100 |

3) Solution Aerosol

| Ingredients | wt. % |
|---|---|
| tiotropium bromide | 0.042 |
| absolute ethanol | 30 |
| purified water | 1.5 |
| anhydrous citric acid | 0.002 |
| TG 134a | to 100 |

### C) Propellant-Free Inhalable Solutions

1) Solution for Use in the RESPIMAT® Nebulizer

| Ingredients | mg/100 mL |
|---|---|
| tiotropium bromide | 148.5 |
| benzalkonium chloride | 10 |
| sodium edetate | 10 |
| hydrochloric acid (aq) | to pH 2.9 |
| water | to 100 mL |

Using the solution in the RESPIMAT® nebulizer leads to a dosage of 10 μg per dose of 1.

2) Solution for Use in the RESPIMAT® Nebulizer

| Ingredients | mg/100 mL |
|---|---|
| tiotropium bromide | 148.5 |
| benzalkonium chloride | 10 |
| hydrochloric acid (aq) | to pH 2.9 |
| water | to 100 mL |

Using the solution in the RESIPMAT® nebulizer leads to a dosage of 10 μg per dose of 1.

3) Solution for Use in the RESPIMAT® Nebulizer

| Ingredients | mg/100 mL |
|---|---|
| tiotropium bromide | 297.1 |
| benzalkonium chloride | 10 |
| sodium edetate | 10 |
| hydrochloric acid (aq) | to pH 2.9 |
| water | to 100 mL |

Using the solution in the RESPIMAT® nebulizer leads to a dosage of 20 μg per dose of 1 and 25 μg/dose of 2.

4) Solution for Use in the RESPIMAT® Nebulizer

| Ingredients | mg/100 mL |
|---|---|
| tiotropium bromide | 297.1 |
| benzalkonium chloride | 10 |
| hydrochloric acid (aq) | to pH 2.9 |
| water | to 100 mL |

Using the solution in the RESPIMAT® nebulizer leads to a dosage of 20 μg per dose of 1.

5) Solution for Use in the RESPIMAT® Nebulizer

| Ingredients | mg/100 mL |
|---|---|
| tiotropium bromide | 148.5 |
| benzalkonium chloride | 8 |
| sodium edetate | 50 |
| hydrochloric acid (aq) | to pH 2.5 |
| water | to 100 mL |

Using the solution in the RESPIMAT® nebulizer leads to a dosage of 10 μg per dose of 1.

6) Solution for Use in the RESPIMAT® Nebulizer

| Ingredients | mg/100 mL |
|---|---|
| tiotropium bromide | 1.5 |
| benzalkonium chloride | 8 |
| sodium edetate | 10 |
| hydrochloric acid (aq) | to pH 2.5 |
| water | to 100 mL |

Using the solution in the RESPIMAT® nebulizer leads to a dosage of 0.1 μg per dose of 1.

7) Solution for Use in the RESPIMAT® Nebulizer

| Ingredients | mg/100 mL |
|---|---|
| tiotropium bromide | 14.9 |
| benzalkonium chloride | 10 |
| sodium edetate | 50 |
| hydrochloric acid (aq) | to pH 3.5 |
| water | to 100 mL |

Using the solution in the RESPIMAT® nebulizer leads to a dosage of 1 μg per dose of 1.

8) Concentrated Solution

| Ingredients | mg/100 mL |
|---|---|
| tiotropium bromide | 1486.1 |
| benzalkonium chloride | 20 |
| sodium edetate | 100 |
| hydrochloric acid (aq) | to pH 3.5 |
| water | to 100 mL |

We claim:

1. A method for reducing the mortality rate in a patient comprising administering to the patient a therapeutically effective amount of a tiotropium salt.

2. The method of claim 1, wherein the tiotropium salt is tiotropium bromide.

3. A method for reducing the respiratory mortality rate in a patient suffering from a lung disease comprising administering to the patient a therapeutically effective amount of a tiotropium salt.

4. The method of claim 3, wherein the tiotropium salt is tiotropium bromide.

5. A method for reducing the respiratory mortality rate in a patient suffering from an obstructive or restrictive lung disease, the method comprising administering to the patient a therapeutically effective amount of a tiotropium salt.

6. The method of claim 5, wherein the tiotropium salt is tiotropium bromide.

7. A method for reducing the respiratory mortality rate in a patient suffering from bronchiectasis, cystic fibrosis, chronic bronchitis, chronic obstructive bronchitis, asthma, chronic obstructive pulmonary disease (COPD), emphysema, or a fibrosing lung disease, the method comprising administering to the patient a therapeutically effective amount of a tiotropium salt.

8. The method of claim 7, wherein the tiotropium salt is tiotropium bromide.

9. A method for reducing the respiratory mortality rate in a patient suffering from chronic (obstructive) bronchitis or chronic obstructive pulmonary disease (COPD) with or without emphysema, the method comprising administering to the patient a therapeutically effective amount of a tiotropium salt.

10. The method of claim 9, wherein the tiotropium salt is tiotropium bromide.
